(19) United States
(12) Patent Application Publication
(10) Pub. No.: US 2016/0145641 A1
(43) Pub. Date: May 26, 2016

(54) STEPWISE DIFFERENTIATION OF STEM CELLS FOR THE PRODUCTION OF EUKARYOTIC MEMBRANE PROTEINS

(71) Applicant: UAB RESEARCH FOUNDATION, Birmingham, AL (US)

(72) Inventors: Lawrence J. DELUCAS, Birmingham, AL (US); John C. KAPPES, Birmingham, AL (US)

(21) Appl. No.: 14/779,238

(22) PCT Filed: Mar. 26, 2014
(86) PCT No.: PCT/US14/31856

§ 371 Date: Sep. 22, 2015

Related U.S. Application Data
(60) Provisional application No. 61/805,794, filed on Mar. 27, 2013.

(51) Int. Cl. C12N 15/85; C07K 14/705; C12N 5/071

(52) U.S. Cl. CPC C12N 15/85; C12N 5/0602; C07K 14/705; C12N 2830/34; C12N 2510/00; C12N 2501/40

(57) ABSTRACT

A method useful for making a eukaryotic membrane protein in vitro may be carried out by (a) propagating in vitro vertebrate stem cells; (b) transforming the vertebrate stem cells in vitro with a heterologous expression vector containing a nucleic acid encoding the eukaryotic membrane protein; and then (c) differentiating the stem cells in vitro into differentiated cells (or photoreceptor-like cells) that express the membrane protein.

### FIELD OF THE INVENTION

The present invention concerns methods and compositions for the production of proteins, particularly membrane proteins such as G-protein coupled receptors (GPRCs), in vitro.

### BACKGROUND OF THE INVENTION

G-protein-coupled receptors (GPCRs) are crucial to cell signal transduction mechanisms and are the object of many pharmaceutical studies, with more than 50% of the medications on the market targeting them.¹ Despite their important biological implications, little structural data on these membrane proteins exists due to their scarce natural abundance. The two widely used techniques to obtain high resolution structures, NMR and X-ray crystallography, require milligram amounts of purified proteins which cannot be obtained using current expression systems (E. coli, yeast, bacculovirus/insect cells, mammalian cells, etc.).^{2,5} It is generally believed that these systems are not equipped with sufficient processing machinery to handle the overexpression of individual membrane proteins as evidenced by the absence of high-affinity binding sites and the presence of misfolded and aggregated proteins.^{1-3,5}

### SUMMARY OF THE INVENTION

The present invention involves a method useful for making a eukaryotic membrane protein in vitro, comprising:

(a) propagating vertebrate stem cells in vitro;

(b) transforming said vertebrate stem cells in vitro with a heterologous expression vector containing a nucleic acid encoding said eukaryotic membrane protein so that said membrane protein is operatively associated in said vertebrate stem cells with a promoter; and then
(c) differentiating said stem cells in vitro into differentiated cells (or photoreceptor-like cells) that express said membrane protein.

Cells or in vitro cultures of cells as described above, and further below, are also an aspect of the invention.

### BRIEF DESCRIPTION OF THE DRAWINGS

FIG. 1. Generating photoreceptor-like cells from hESCs, testing growth on both poly-D-lysine/laminin/fibronectin-coated and C3/VITA 6-well plates in the presence or absence of NM23 antibody; using the ROCK inhibitor Y-27632 plus Wnt and Nodal antagonists DKK-1 and Lefty-A during the first 15-21 days of differentiation to produce retinal progenitor cells, and retinoic acid and taurine during differentiation days 91-150 to generate photoreceptor-like cells from hESCs.

### DETAILED DESCRIPTION OF NON-LIMITING EMBODIMENTS

In some embodiments of the foregoing, the propagating step includes the step of contacting said stem cells with at least one MUC1*-activating ligand (e.g., dimeric NM23; bivalent anti-MUC1 * antibodies) by an amount sufficient to stimulate the growth and/or viability of said cells.

In some embodiments, the propagating step is carried out in the absence of feeder cells, and/or in the absence of fibroblast growth factor.

In some embodiments, the transforming step is carried out with a lentiviral vector.

In some embodiments, the promoter is a heterologous or homologous (or endogenous or exogenous) rhodopsin promoter.

In some embodiments, the transforming step further comprises the step of knocking down homologous rhodopsin expression in said cells.

In some embodiments, the differentiating step comprises contacting said vertebrate stem cells to at least one photoreceptor differentiation factor (e.g., retinoic acid, taurine, sonic hedgehog protein (Shh), 3-isobutyl-1-methylxanthine (IBMX), etc.).

In some embodiments, the vertebrate stem cells are retinal stem cells.

In some embodiments, the vertebrate stem cells are avian, amphibian, reptile, or mammalian cells.

In some embodiments, the vertebrate stem cells are embryonic stem cells (or amniotic fluid stem cells, placental stem cells, adiopose stem cells, induced pluripotent stem cells, etc.)

In some embodiments, the eukaryotic membrane protein is a plant (e.g., vascular plant such as an angiosperm or gymnosperm, or monocot or dicot), animal (e.g., veterbrate such as fish, amphibian, reptile, avian, or mammalian species), protozoal, or fungal (e.g., yeast, mold, etc.) protein.

In some embodiments, the eukaryotic membrane protein is a receptor, ion channel (e.g., voltage gated, ligand gated, etc.), ion pump, or carrier protein.

In some embodiments, the membrane protein is a G protein-coupled receptor (GPCR), such as a Class A (or 1; Rhodopsin-like family), Class B (or 2; Secretin receptor family), Class C (or 3: metabotropic glutamate/pheromone family), Class D (or 4; fungal mating pheromone receptor family), Class E (or 5; cyclic AMP receptor family), or Class F (or 6, frizzled/smoothened family) GPRC. Examples thereof include but are not limited to dopamine D1 receptor, a dopamine D2 receptor, a dopamine D3 receptor, a dopamine D5 receptor, a histamine 1 receptor, a cysteinyl leukotriene receptor 1, a cysteinyl leukotriene receptor 2, an opioid receptor, a muscarinic receptor, a serotonin receptor, a beta2-adrenergic receptor or a metabotropic glutamate 4 receptor. G protein coupled receptors and uses thereof are known and described in, for example, U.S. Pat. Nos. 8,354,241 and 8,329,432.

Methods of the invention may further comprise the step of (d) collecting, enriching, isolating and/or purifying the membrane protein from the differentiated cells, which may be carried out in accordance with known techniques such as cell lysis, filtration, chromatography, centrifugation, etc., including combinations thereof.

Such membrane proteins can be used for any suitable purpose, including but not limited to binding assays, and crystallization for subsequent structural analysis as described in U.S. Pat. No. 8,329,432.

### EXPERIMENTAL

### Stepwise Differentiation of Human Embryonic Stem Cells into Mature Photoreceptors: Creation of a GPCR Membrane Protein Factory

Nature has posed an ideal solution to the overexpression problem noted in the “Background of the Invention” above: Rhodopsin.

High quantities of the GPCR rhodopsin are properly folded and expressed in in the vertebrate retina—about 10⁷ molecules per day per photoreceptor.³ Rhodopsin is continuously synthesized within the rod inner segments and transferred into the outer segments to await phototransduction, accumulating until more than 98% of their protein content is rhodopsin. The chaperone machinery that facilitates rhodopsin folding in such high fidelity is extremely efficient in photoreceptor cells, making them an excellent potential overexpression system for GPCRs.³ Although there is resounding evidence to support the retina's biochemical machinery is capable of producing properly folded, biologically functional GPCRs other than rhodopsin in transgenic animals, the yield is extremely low and therefore not cost effective.^{1,4,5} To address these GPCR expression system dilemmas, a novel method has been developed to generate photoreceptor-like cells from human derived cultures, keeping in mind the ultimate goal of expressing large quantities of GPCR for structural studies.

This innovative technique will involve the expansion of stem cells in the presence of exogenous factors on proprietary substrates to cultivate photoreceptor-like cells. As photoreceptors and their progenitors are terminally differentiated cell lines and cannot multiply, one must start at the stem cell level and direct differentiation towards retinal cells, Using growth factors as biomimetic signaling cues, stem cells can be directed to differentiate into retinal progenitors then photoreceptor-like cells. Researchers have identified particular growth factor combinations that can increase the portion of retinal stem cells to differentiate towards photoreceptor-like (Rhodopsin+) cells.⁶⁻⁸ Osakada (2008) generated photoreceptor-like cells from human embryonic stem cells (hESCs) by culturing them in suspension with Wnt and Nodal pathway inhibitors Dkk-1 and Lefty-A then guiding differentiation towards a photoreceptor fate by adding retinoic acid and taurine.⁶ Other studies have directed hESCs to differentiate into retinal progenitors by growing them in the presence of basic fibroblast growth factor (bFGF) without Dkk-1 and Noggin antagonists and influencing them to differentiate towards photoreceptor-like cells by using chemically defined neural induction medium.⁸ Presently, the standard for hESC growth requires combination of bFGF as well as other undefined growth factors secreted from fibroblast feeder cells.¹⁰⁻¹² However, growing hESCs in these optimized environments only yields about 65-75% undifferentiated, pluripotent stem cells.¹⁰ This reduced yield becomes problematic as cells that have started to differentiate secrete factors that encourage neighboring cells to differentiate as well. Accordingly, advances have been made in determining growth factors that prevent differentiation from occurring and negate the need to add fibroblast based factors to cell cultures.

Recently, it was shown that cancerous cells, as well as stem cells, present a proteolytic degradation product of the Type 1 membrane glycoprotein mucin 1 (MUC1), termed MUC1*.^{9,10} MUC1* has growth factor receptor-like activity that stimulates cell growth via mitogen-activated protein (MAP) kinase signaling pathways.¹⁰ The natural ligand of MUC1* is NM23, which is released by tumor cells and stem cells and binds to the protein with nanomolar affinity.⁹ The addition of NM23 during culture stimulates growth and inhibits apoptosis of cells that present MUC1*, as evidenced in Hikita (2008). In their study, MUC1* increased cell growth several fold, enabled growth of stem cells without feeder cells or FGF, and prevented spontaneous differentiation.¹⁰ The proposed studies will compare the differentiation efficiency of this novel MUC1* culture system to the standard method involving bFGF to evaluate the potential of this approach to produce of milligram to gram amounts of GPCRs for structural studies.

### OBJECTIVES

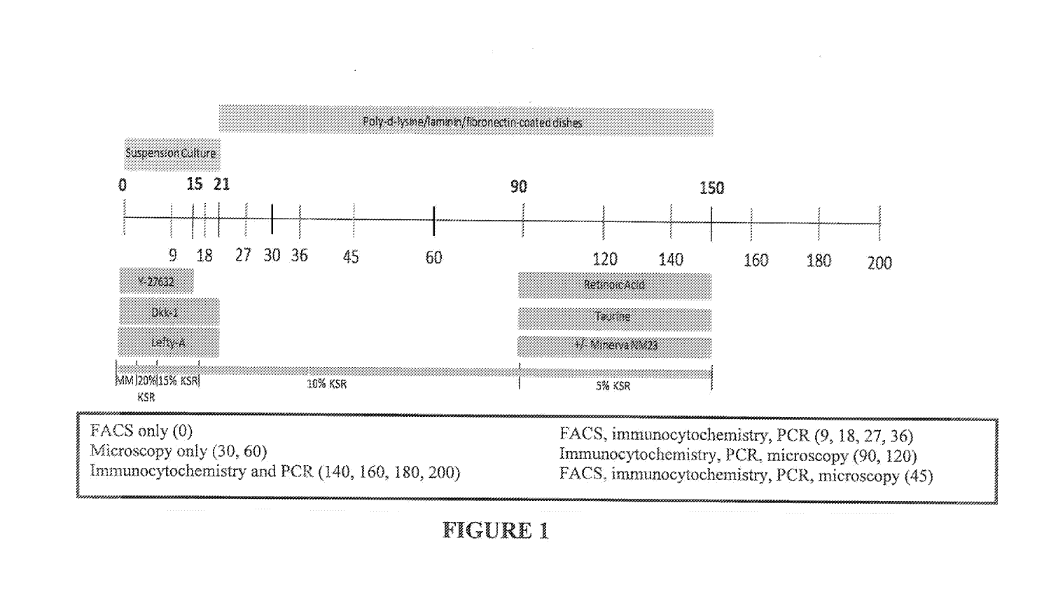

Because MUC1 shifts between the ON/OFF state, functioning as a growth factor in the clipped form and representing the quiescent state in the full-length form, this study will focus on whether the growth and differentiation of hESCs and retinal progenitors are mediated by the oncoprotein MUC1 as it transitions between a cleaved (MUC1*) and uncleaved (MUC1) state. The use of NM23, in combination with retinoic acid and taurine, as a media supplement will stimulate growth and inhibit apoptosis of cells that present MUC1* and alter the differentiation of hESCs towards a photoreceptor lineage. These photoreceptor-like cells will then be transduced with lentiviral expression vectors designed to downregulate rhodopsin production and upregulate production of another membrane protein. Although the overall goal of research is to generate large quantities of GPCR protein, this portion of the study will test if our innovative cell culture system, a novel combination of antibody coatings, NM23 growth factor, and other retinal signaling cues established in literature, will generate a high yield of photoreceptor-like cells from human embryonic stem cells. Specifically, it aims to generate photoreceptor-like cells from hESCs, testing growth on poly-D-lysine/laminin/fibronectin-coated surfaces in the presence or absence of NM23 antibody; using the ROCK inhibitor Y-27632 plus Wnt and Nodal antagonists DKK-1 and Lefty-A during the first 15-21 days of differentiation to produce retinal progenitor cells, and retinoic acid and taurine during differentiation days 91-150 to generate photoreceptor-like cells from hESCs (FIG. 1).

Throughout this study, the progression of hESCs—from pluripotent stem cells developing into the early eye field and transitioning to retinal progenitors and photoreceptor-like cells—will be assessed by measuring key transcription factors Oct4, Rx, Pax6, Chx10, Mitf, Crx, Rhodopsin, Recoverin, Red/Green and Blue opsin. Oct4 is considered to be one of the transcription factors vital to the propagation and regulation of hESCs, making it a key marker for cell pluripotency.⁷ Expression of the transcription factor Rx suggests early eye field development and coincides with Pax6 expression in neural retinal progenitors; this Pax6 expression steadily diminishes as the neural retina develops.¹³ Mitf gene expression is an important indicator of RPE development; however, in neuroretinal progenitors, it is suppressed by Chx10 in eye formation.¹⁴ Crx is a retinal progenitor marker, whose expression is necessary for the proper advancement, specialization, and maintenance of rods and cones.^{7,13} The presence of photoreceptors is determined by measuring for opsin GPCRs: rods utilize rhodopsin, while cone utilize short-wavelength (blue), medium-wavelength (green) or long-wavelength (red) sensitive opsins. Photoreceptors are further defined by the incidence of Recoverin, a genetic marker acknowledged for characterizing mature and developing photoreceptors.⁷

### MATERIALS AND METHODS

The human embryonic stem cells (hESCs) are grown in 0.1% gelatin coated 6-well plates on a feeder layer of mitomycin-C-inactivated, primary mouse embryonic fibroblasts (PMEFs; Millipore) and maintained in maintenance media: DMEM/F12/GlutaMAX media (Invitrogen) supplemented with 20% knockout serum replacement (Invitrogen), 1% MEM nonessential amino acids (Invitrogen), and 0.18% β-mercaptoethanol (Invitrogen), with 4 ng/mL basic human fibroblast growth factor (StemGent). Media is changed every day until confluent (˜every 5-7 days) before passing the cells onto fresh gelatin-coated PMEF plates. Plates are checked daily and all differentiated colonies are dissected out before passaging. All cultures are incubated at 37° C. in a 90% humidified atmosphere with 5% CO₂.

To initiate the photoreceptor differentiation process, the hESCs will propagate on low cell binding plates (floating culture) in maintenance media plus Wnt and Nodal antagonists DKK-1 and Lefty-A (R&D Systems) for the first 21 days of differentiation and rho-associated coil kinase inhibitor (ROCKi) Y-27632 (Sigma-Aldrich) during the first 15 days of differentiation to increase cell survival. The hESC aggregates will then be seeded onto poly-d-lysine/laminin/fibronectin to differentiate over the next 70 days, approximately. Retinoic acid and taurine (Sigma-Aldrich), with and without supplemented NM23 (Minerva Biotechnology), will be added to media near differentiation day 90 to develop photoreceptor-like cells by differentiation day 150. Media will be replaced every three days during floating culture conditions and everyday once attached to coated plates.

The efficacy of the photoreceptor-like cells to replicate native structure, protein content, and gene expression in vitro will be determined by: fluorescence-activated cell sorting (FACS) analysis, fluorescent staining, phase contrast microscopy, and polymerase chain reaction (PCR) to test for retinal-specific genetic markers according to intervals found in nature.

### BROADER IMPACTS

These photoreceptor-like cells will be transduced with lentiviral expression vectors specifically designed to knock-down rhodopsin production with a gene inserted for an alternative GPCR protein. Ultimately, this project will provide the scientific community with a method to produce large quantities of GPCRs for structural studies and pharmaceutical research and enable the study and formulation of retinal disease models to accelerate the use of progenitors in human transplant studies as a form of therapy.

### REFERENCES

1. Sarramegna et al. Cellular and Molecular Life Sciences (2003).
2. McCusker et al. Biotechnology Progress. (2007).
3. Chapple and Cheetham. Journal of Biological Chemistry (2003)
4. Opekarova and Tanner. Biochmica et Biophysica Acta (2003).
5. Sarramegna et al. Cellular and Molecular Life Sciences (2006).
6. Osakada et al. Nature Biotechnology (2008).
7. Vugler et al. PNAS. (2009).
8. Sheedlo et al., In Vitro Cell Dev. Biol., 43:361-370 (2007); Ezeonu et al., DNA and Cell Biology, 22: 607-620 (2003).
9. Mahanta et al. PLoS ONE (2008).
10. Hikita et al. PLoS ONE (2008).
11. Richards et al. Nature Biotechnology (2002).
12. Xu et al. Stem Cells (2005)
13. Ikeda et al. PNAS (2005)
14. Horsford et al. Development (2005).

1. A method useful for making a eukaryotic membrane protein in vitro, comprising:
(a) propagating in vitro vertebrate stem cells;
(b) transforming said vertebrate stem cells in vitro with a heterologous expression vector containing a nucleic acid encoding said eukaryotic membrane protein so that said membrane protein is operatively associated in said vertebrate stem cells with a promoter; and then
(c) differentiating said stem cells in vitro into differentiated cells or photoreceptor-like cells that express said membrane protein.

2. The method of claim 1, wherein said propagating step includes the step of contacting said stem cells to at least one MUC1* activating ligand by an amount sufficient to stimulate the growth and/or viability of said cells.

3. The method of claim 1 wherein said propagating step is carried out in the absence of feeder cells, and/or in the absence of fibroblast growth factor.

4. The method of claim 1, wherein said transforming step is carried out with a lentiviral vector.

5. The method of claim 1, wherein said promoter is a heterologous or homologous rhodopsin promoter.

6. The method of claim 1, wherein said transforming step further comprises the step of knocking down homologous rhodopsin expression in said cells.

7. The method of claim 1, wherein said differentiating step comprises contacting said vertebrate stem cells to at least one photoreceptor differentiation factor.

8. The method of claim 1, wherein said vertebrate stem cells are retinal stem cells.

9. The method of claim 1, wherein said vertebrate stem cells are avian, amphibian, reptile, or mammalian cells.

10. The method of claim 1, wherein said vertebrate stem cells are embryonic stem cells.

11. The method of claim 1, wherein said eukaryotic membrane protein is a plant, animal, protozoal, or fungal protein.

12. The method of claim 1, wherein said eukaryotic membrane protein is a receptor, ion channel, ion pump, or carrier protein.

13. The method of claim 1, wherein said membrane protein is a G protein-coupled receptor (GPCR).

14. The method of claim 1, further comprising the step of:
(d) collecting said membrane protein from said differentiated cells.

15. A cell or in vitro culture of cells produced by the method of claim 1.
